(19) United States
(12) Patent Application Publication
(10) Pub. No.: US 2024/0324992 A1
(43) Pub. Date: Oct. 3, 2024

(54) ULTRASOUND PROBE MANUFACTURING METHOD

(71) Applicant: VERMON SA, Tours (FR)

(72) Inventor: Cyril MEYNIER, Tours (FR)

(21) Appl. No.: 18/609,363

(22) Filed: Mar. 19, 2024

(30) Foreign Application Priority Data
Apr. 3, 2023 (FR) 23/03295

(51) Int. Cl. A61B 8/00; A61B 8/12; A61N 7/00

(52) U.S. Cl. CPC A61B 8/445; A61B 8/12; A61B 8/4494; A61N 7/00; A61N 2007/0078

(57) ABSTRACT

A method of manufacturing an ultrasound probe, including the following steps: a) transferring, onto a flexible interconnection substrate, a chip including a plurality of ultrasound transducers, the chip including a front side including at least one electrical connection pad and a back side; b) folding a portion of the flexible interconnection substrate, so that the flexible interconnection substrate extends over at least a portion of the back side of the chip and over at least a portion of the front side of the chip, said at least one electrical connection pad of the front side of the chip being connected to at least one corresponding electrical connection pad of the interconnection substrate; and c) cutting the chip into a plurality of elementary chips, each including one or a plurality of ultrasound transducers.

### FIELD OF THE INVENTION

The present disclosure generally concerns the manufacturing of ultrasound probes, and particularly aims at the manufacturing of ultrasound probes of small dimensions intended to be mounted on a catheter, for example for diagnostic or intravascular ultrasound treatment applications.

### DESCRIPTION OF RELATED ART

It has already been provided to integrate small ultrasound probes into a catheter intended to be introduced into a human or animal patient's body, for example for diagnostic or intravascular ultrasound treatment applications.

It would be desirable to have a method of manufacturing such a probe, this method at least partially overcoming some of the disadvantages of known methods.

### BRIEF SUMMARY OF THE INVENTION

An embodiment provides a method of manufacturing an ultrasound probe, comprising the following steps:
a) transferring, onto a flexible interconnection substrate, a chip comprising a plurality of ultrasound transducers, the chip comprising a front side comprising at least one electrical connection pad and a back side;
b) folding a portion of the flexible interconnection substrate, so that, at the end of step b), the flexible interconnection substrate extends over at least a portion of the back side of the chip and over at least a portion of the front side of the chip, said at least one electrical connection pad of the front side of the chip being connected to at least one corresponding electrical connection pad of the interconnection substrate; and
c) cutting the chip into a plurality of elementary chips, each comprising one or a plurality of ultrasound transducers.

According to an embodiment, step c) is implemented after step b).

According to an embodiment, step c) is implemented before step b).

According to an embodiment, at step a), the chip has its back side facing the surface of the interconnection substrate comprising said at least one electrical connection pad of the interconnection substrate.

According to an embodiment, at step a), the chip has its front side facing the surface of the interconnection substrate comprising said at least one electrical connection pad of the interconnection substrate.

According to an embodiment, at the end of step c), the elementary chips remain mechanically coupled to one another by an uncut strip of the flexible interconnection substrate.

According to an embodiment, during step b), a cylindrical rod is positioned on the interconnection substrate to control the bending of the interconnection substrate during the folding.

According to an embodiment, the method comprises, after steps a), b), and c), a step of bending of the interconnection substrate according to a desired shape of the probe, for example a cylindrical shape.

According to an embodiment, the ultrasound transducers are transducers of CMUT or PMUT type.

According to an embodiment, the chip further comprises at least one electrical connection pad on its back side.

Another embodiment provides an ultrasound probe comprising a flexible interconnection substrate and a plurality of elementary chips bonded to the flexible interconnection substrate, each elementary chip comprising one or a plurality of ultrasound transducers, each elementary chip comprising a front side comprising at least one electrical connection pad and a back side,

wherein a portion of the flexible interconnection substrate is folded over the elementary chips, so that the flexible interconnection substrate extends over at least a portion of the back side of each elementary chip and over at least a portion of the front side of each elementary chip, said at least one electrical connection pad of the front side of each elementary chip being connected to a corresponding electrical connection pad of the interconnection substrate.

### BRIEF DESCRIPTION OF THE DRAWINGS

The foregoing features and advantages, as well as others, will be described in detail in the rest of the disclosure of specific embodiments given by way of illustration and not limitation with reference to the accompanying drawings, in which:

FIG. 1 is a very simplified perspective view of an example of an ultrasound probe;

FIG. 2A, FIG. 2B, and FIG. 2C illustrate steps of an example of a method of manufacturing an ultrasound probe according to an embodiment; and

FIG. 3A and FIG. 3B illustrate steps of a variant of a method of manufacturing an ultrasound probe according to an embodiment.

### DETAILED DESCRIPTION OF EXEMPLARY EMBODIMENTS OF THE INVENTION

Like features have been designated by like references in the various figures. In particular, the structural and/or functional features that are common among the various embodiments may have the same references and may dispose identical structural, dimensional and material properties.

For the sake of clarity, only the steps and elements that are useful for the understanding of the described embodiments have been illustrated and described in detail. In particular, the forming of the ultrasound transducers of the described probes has not been detailed, the described embodiments being compatible with all or most known ultrasound transducer structures. Further, the forming of the circuits for controlling the ultrasound transducers has not been detailed, the embodiments described being compatible with usual ultrasound transducer control circuits, or the forming of the control circuits being within the abilities of those skilled in the art based on the indications of the present disclosure.

Unless specified otherwise, when reference is made to two elements connected together, this signifies a direct connection without any intermediate elements other than conductors, and when reference is made to two elements coupled together, this signifies that these two elements can be connected or they can be coupled via one or more other elements.

In the following description, when reference is made to terms qualifying absolute positions, such as terms “front”, “back”, “top”, “bottom”, “left”, “right”, etc., or relative positions, such as terms “above”, “under”, “upper”, “lower”, etc., or to terms qualifying directions, such as terms “horizontal”, “vertical”, etc., it is referred, unless specified otherwise, to the orientation of the drawings.

Unless specified otherwise, the expressions “about”, “approximately”, “substantially”, and “in the order of” signify plus or minus 10%, preferably of plus or minus 5%.

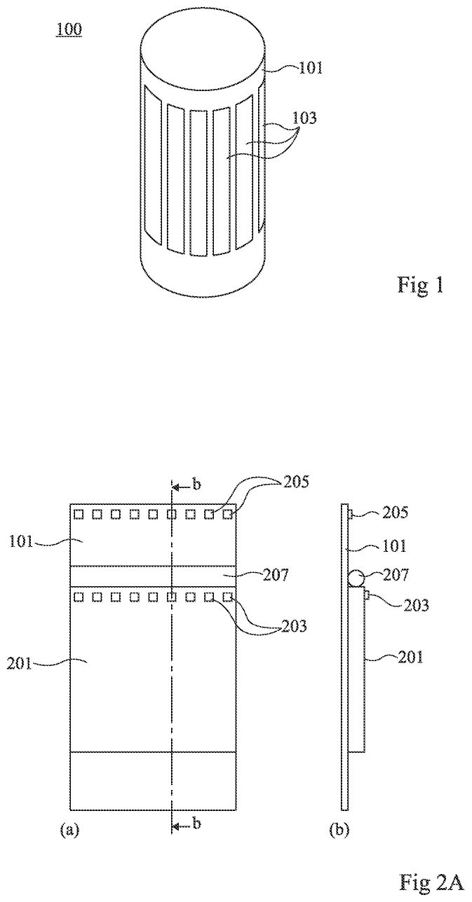

FIG. 1 is a very simplified perspective view of an example of an ultrasound probe 100.

Probe 100 comprises a flexible interconnection substrate 101 having a plurality of elementary chips 103, each comprising one or a plurality of ultrasound transducers, not detailed in the drawings, bonded thereto. As an example, each elementary chip 103 is an ultrasonic wave transceiver chip, also called “ultrasonic array”, comprising one or a plurality of elementary ultrasound transducers, for example aligned along an axis parallel to a longitudinal axis of the probe or arranged in a matrix on the plane of the array.

Flexible interconnection substrate 101 is wound around a cylinder (not shown in the drawing), for example having a circular cross-section, so as to obtain a probe of generally cylindrical shape, for example of small dimensions, for example with a diameter in the range from 1 to 5 mm. The elementary chips form facets adapted to emitting ultrasounds towards the outside of the cylinder, in radial directions. In this example, the elementary chips have the shape of parallel rectangular strips, extending parallel to the central axis of the cylinder. As an example, the probe comprises at least two, preferably at least four, more preferably at least eight, elementary chips 103, for example evenly distributed around the cylinder.

Each chip comprises electrical connection pads (not shown in FIG. 1) connected to corresponding electrical connection elements (not shown in FIG. 1) of interconnection substrate 101.

Interconnection substrate 101 is, for example, a flexible printed circuit board, comprising electrically-conductive interconnection elements, for example metal tracks and pads, for example made of copper, formed on a flexible dielectric support, for example made of a polymer material, for example a polyimide. One or a plurality of electrically conductive wires, not detailed in the drawing, may electrically connect interconnection substrate 101 to electronic circuits external to the probe, to control elementary chips 103.

To form such a probe, a possibility is to bond and electrically connect on a connection surface of interconnection substrate 101 a single monolithic chip, comprising all the ultrasound transducers of the probe, and then to cut the chip into a plurality of elementary chips 103, each comprising one or a plurality of ultrasound transducers. At the end of the cutting step, each elementary chip 103 remains bonded and electrically connected to interconnection substrate 101. Substrate 101 may then be wound around a cylinder, to give the desired shape to the probe.

The electrical connection between the monolithic chip and interconnection substrate 101 may be performed by means of conductive wires coupling the connection surface of the interconnection substrate to the front side of the chip, opposite to the interconnection substrate. An encapsulating resin, such as an epoxy resin, may be provided to protect the connection wires. This wire bonding method however has disadvantages. In particular, the conductive wires and, where applicable, the encapsulating resin, result in increasing the diameter of the probe. Further, there is a risk of damaging the conductive wires during the step of cutting of the monolithic chip into individual chips 103.

Alternatively, the monolithic chip and interconnection substrate 101 may be assembled by a so-called “flip-chip” method. In this case, the chip comprises electrical connection pads on its back side, that is, on its surface opposite to the ultrasonic wave transceiver surface. These pads are directly positioned on top of and in contact with corresponding electrical connection pads located on the connection surface of interconnection substrate 101. Thus, the bulk is decreased with respect to a conductive wire connection, and risks of damaging the electrical connections during the step of cutting of the monolithic chip are decreased. However, a disadvantage of this solution is that the manufacturing cost of the transducer chip is relatively high, due to the need to integrate conductive vias crossing the chip substrate to couple the ultrasound transducers to the electrical connection pads on the back side of the chip. This is particularly true in the case of transducers in MEMS (Micro Electro Mechanical System) technology.

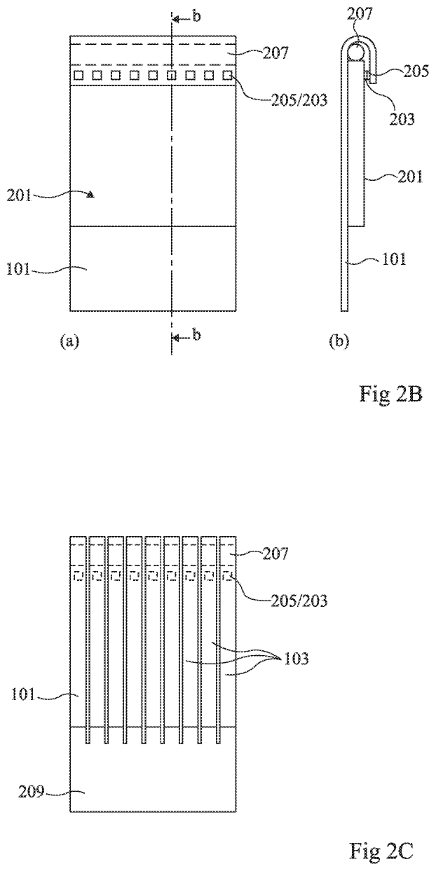

FIGS. 2A, 2B, and 2C illustrate successive steps of an example of a method of manufacturing an ultrasound probe according to an embodiment.

FIG. 2A illustrates the assembly obtained at the end of a step of transferring a monolithic chip 201 onto a connection surface of a flexible interconnection substrate 101.

FIG. 2A shows a front view (a) of the assembly, and a cross-section view (b) along plane b-b of view (a).

At this stage of the method, the flexible interconnection substrate is unwound and positioned, for example, on a flat support, not shown.

Chip 201 for example has a rectangular shape. Chip 201 comprises a plurality of ultrasound transducers corresponding to all the probe transducers. The transducers (not detailed in the drawings) are, for example, transducers of CMUT (capacitive membrane transducer) type or transducers of PMUT (piezoelectric membrane transducer) type. The described embodiments are however not limited to this specific case and more generally apply to all types of ultrasound transducers. The forming of chip 201 has not been detailed, the described embodiments being compatible with all or most known ultrasound transducer chip manufacturing methods, or the forming of such a chip being within the abilities of those skilled in the art based on the indications of the present disclosure. Chip 201 comprises a front side corresponding to the ultrasonic wave transceiver surface, and a back side opposite to its front side. Chip 201 comprises electrical connection pads 203 arranged on its front side.

Interconnection substrate 101 comprises a connection surface comprising electrical connection pads 205 intended to be respectively connected to the electrical connection pads 203 of chip 201.

In this example, during the transfer of chip 201, the back side of the chip 201 faces the connection surface of interconnection substrate 101. The back side of chip 201 is, for example, bonded to the connection surface of interconnection substrate 101 by an adhesive layer, not shown in the drawing. A portion of interconnection substrate 101 comprising electrical connection pads 205 is not covered with chip 201. In the shown example, interconnection substrate 101 covers the entire back side of the chip. Thus, in this example, the surface area of interconnection substrate 101 is greater than the surface area of chip 201. The described embodiments are however not limited to this specific case. As a variant, interconnection substrate 101 may cover a portion only of the back side of chip 201.

The electrical connection pads 205 of interconnection substrate 101 are arranged according to an arrangement substantially identical to that of the electrical connection pads 203 of chip 201. Electrical connection pads 205 are arranged in a portion of interconnection substrate 101 designed to be folded onto the front side of chip 201, so as to place pads 203 and 205 respectively in front of each other.

In the shown example, a rod 207 of generally cylindrical shape, for example of circular cross-section, is arranged on the connection surface of interconnection substrate 101, for example against an edge of chip 201. Rod 207 is arranged, in top view, between an edge of chip 201 and the portion of interconnection substrate 101 comprising connection pads 205, along the folding axis of substrate 101. Rod 207, which is optional, advantageously enables to control the radius of curvature of interconnection substrate 101 during the folding thereof onto the front side of chip 201, limiting the risk of breakage of the metal tracks of substrate 101 during the folding. Rod 207 is for example a wire, for example made of nylon.

FIG. 2B illustrates the assembly obtained at the end of a step of folding of the portion of interconnection substrate 101 comprising connection pads 205 onto the front side of chip 201, in such a way as to position the connection pads 205 of the substrate in front of the connection pads 203 of the chip.

FIG. 2B shows a front view (a) of the assembly, and a cross-section view (b) along plane b-b of view (a).

In this example, the substrate is wrapped around rod 207 during the folding step. A tool having a shape complementary to rod 207, optional and not shown in FIGS. 2A and 2B, may be used to clamp and hold substrate 101 in position around rod 207 during this step and the following.

During this step, the connection pads 205 of the substrate are respectively bonded and electrically connected to the corresponding connection pads 203 of chip 201. The bonding and the electrical connection of pads 203 to pads 205 may be achieved by any adapted method, for example by soldering, brazing, gluing by means of a conductive paste or glue, by means of an anisotropic adhesive conductive film (ACF), etc.

Preferably, flexible interconnection substrate 101 does not cover the entire front side of chip 201. In particular, the ultrasound transceiver surface of the chip is preferably not covered by substrate 101, so as not to interfere with the propagation of ultrasonic waves. As a variant, the front side of chip 201 is totally covered by interconnection substrate 101 after the folding step.

FIG. 2C is a front view illustrating the assembly obtained at the end of a step of cutting of the chip into a plurality of elementary chips 103, each comprising one or a plurality of ultrasound transducers. Each elementary chip 103 comprises at least one electrical connection pad 203 connected to a corresponding electrical connection pad 205 of flexible interconnection substrate 101.

The cutting may be carried out by any known method for cutting a chip into a plurality of elementary chips, for example by means of a saw or by laser cutting.

In this example, elementary chips 103 are singulated along cutting lines parallel to the central axis of the final cylindrical ultrasound probe.

At least one strip 209 of flexible interconnection substrate 101 is not cut during this step. At the end of the cutting step, elementary chips 103 remain mechanically coupled to each other by said strip 209.

In the shown example, chip 201, rod 207 (optional), and interconnection substrate 101 are entirely cut along the cutting paths. Thus, the uncut strip 209 is not located in front of chip 201. As a variant (not shown), only the portion of interconnection substrate 101 covering the front side of chip 201, rod 207 (optional), and chip 201 are cut. In other words, the portion of the interconnection substrate arranged on the back side of chip 201 is not cut and forms the strip mechanically holding elementary chips 103.

At the end of the cutting step, substrate 101 may be rolled up to form a cylindrical probe of the type described in relation with FIG. 1.

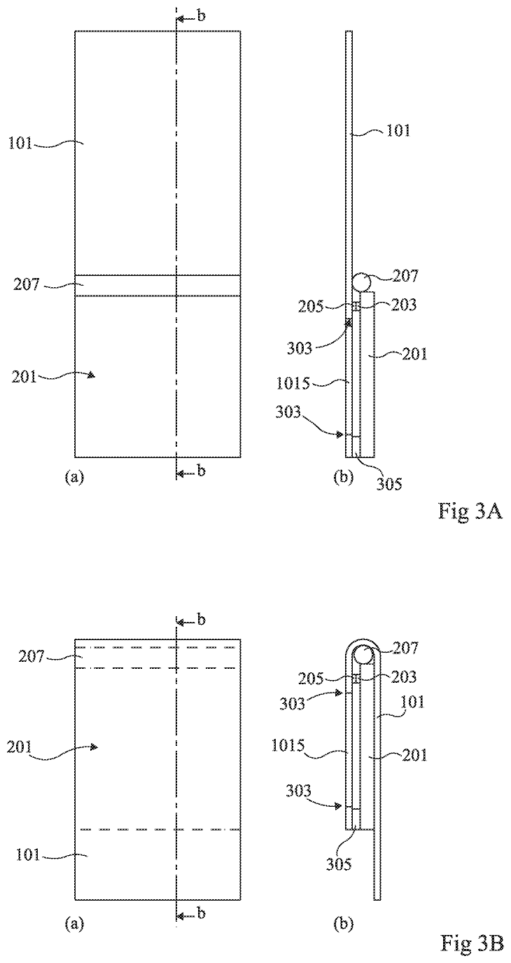

FIGS. 3A and 3B illustrate successive steps of another example of a method of manufacturing an ultrasound probe according to an embodiment.

FIG. 3A illustrates the assembly obtained at the end of a step of transfer of a monolithic chip 201 onto a connection surface of a flexible interconnection substrate 101.

FIG. 3A comprises a front view (a) of the assembly, and a cross-section view (b) along plane b-b of view (a).

The step of FIG. 3A differs from the step of FIG. 2A essentially in that, in the example of FIG. 3A, during the transfer, chip 201 has its front side facing the connection surface of flexible interconnection substrate 101.

Thus, the electrical connection pads 203 of the chip are placed directly in front of the electrical connection pads 205 of substrate 101 and electrically connected to pads 205.

An advantage of this variant is that the connection of pads 203 and 205 is easier to achieve when flexible substrate 101 is fully unwound and planar than after the folding of substrate 101 as described in the previous example. In particular, this makes the application of a pressure to the pads during the bonding easier.

In the shown example, the front side of chip 201 is entirely covered by substrate 101. In this (non-limiting) example, a portion 101S of substrate 101 covering the ultrasonic transceiver area of chip 201, called sacrificial portion, is precut along a cutting line 303, to allow its removal at a subsequent step.

Further, in the (non-limiting) shown example, substrate 101 comprises, for example in its sacrificial portion 101S, an extra thickness 305 projecting towards chip 201. Extra thickness 305 has, for example, a height substantially equal to the cumulated height of the projecting portions of connection pads 203 and 205. During the step of bonding of the pads 203 of the chip to the pads 205 of the substrate, chip 201 bears, by its front side, on extra thickness 305. This enables to compensate for the height of the pads and to keep chip 201 substantially parallel to substrate 101 during this step.

FIG. 3B illustrates the assembly obtained at the end of a step of folding of interconnection substrate 101 onto the back side of chip 201.

In this example, the substrate is wound around rod 207 (optional) during the folding step.

The back side of chip 201 is for example bonded to the connection surface of interconnection substrate 101 by an adhesive layer, not shown in the drawing.

At the end of this step, the sacrificial portion 101S of interconnection substrate 101, and, where applicable, extra thickness 305, may be removed from the front side of the chip.

The rest of the method is, for example, identical or similar to what has been described hereabove.

Various embodiments and variants have been described. Those skilled in the art will understand that certain features of these various embodiments and variants may be combined, and other variants will occur to those skilled in the art. In particular, the described embodiments are not limited to the specific example described hereabove of application to a cylindrical ultrasound probe, but may be applied to other non-planar probe shapes, subject to possible adaptations within the abilities of those skilled in the art.

Further, the described embodiments are not limited to the example of application indicated hereabove to the forming of intravascular ultrasound probes, but more generally apply to the forming of all types of ultrasound probes having non-planar shapes, for example of small dimensions, for medical or non-medical applications.

Further, although examples of embodiment where flexible substrate 101 is folded onto the front or back side of monolithic chip 201 before the cutting of chip 201 into individual chips 103 have been described hereabove, the described embodiments are not limited to this specific case. As a variant, the cutting of monolithic chip 201 may be performed after the bonding thereof to flexible interconnection substrate 101, but before the step of folding of a portion of the substrate 101 onto the surface of the chip opposite to its transfer surface. In particular, those skilled in the art will be capable of adapting the example of FIGS. 2A to 2C by implementing the cutting of chip 201 between the transfer step of FIG. 2A and the folding step of FIG. 2B. Similarly, those skilled in the art will be capable of adapting the example of FIGS. 3A and 3B by implementing the cutting of chip 201 between the transfer step of FIG. 3A and the folding step of FIG. 3B.

Further, in the two examples of implementation described in relation with the drawings, connections have been described on the front side only (ground and hot spot of each chip). It is however possible to provide electrical connections on the two main surfaces of each chip.

As an example, a ground connection may be provided on the front side, and transferred to the folded portion of the flexible substrate. Electrodes of connection to the ultrasound transducers may be provided on the back side, respectively connected to corresponding pads of the flexible substrate.

According to another example, a ground connection may be provided on the back side, connected to a metal plane, for example made of copper, on the flexible interconnection substrate. Electrodes of connection to the ultrasound transducers may be provided on the front side, connected to the folded portion of the flexible interconnection substrate.

What is claimed is:

1. A method of manufacturing an ultrasound probe, comprising the following steps:
a) transferring, onto a surface of a flexible interconnection substrate, a chip comprising a plurality of ultrasound transducers, the chip comprising a front side comprising at least one electrical connection pad and a back side;
b) folding a portion of the flexible interconnection substrate, so that, at the end of step b), said surface of the flexible interconnection substrate extends over and is bonded to at least a portion of the back side of the chip and at least a portion of the front side of the chip, said at least one electrical connection pad on the front side of the chip being connected to at least one corresponding electrical connection pad of said surface of the interconnection substrate; and c) cutting the chip into a plurality of elementary chips, each comprising one or a plurality of ultrasound transducers.

2. The method according to claim 1, wherein step c) is implemented after step b).

3. The method according to claim 1, wherein step c) is implemented before step b).

4. The method according to claim 1, wherein, at step a), the chip has its back side facing the surface of the interconnection substrate comprising said at least one electrical connection pad of the interconnection substrate.

5. The method according to claim 1, wherein, at step a), the chip has its front side facing the surface of the interconnection substrate comprising said at least one electrical connection pad of the interconnection substrate.

6. The method according to claim 1, wherein, at the end of step c), the elementary chips remain mechanically coupled to one another by an uncut strip of the flexible interconnection substrate.

7. The method according to claim 1, wherein, at step b), a cylindrical rod is positioned on the interconnection substrate to control the bending of the interconnection substrate during the folding.

8. The method according to claim 1, comprising, after steps a), b), and c), a step of bending of the interconnection substrate according to a desired shape of the probe, for example a cylindrical shape.

9. The method according to claim 1, wherein the ultrasound transducers are transducers of CMUT or PMUT type.

10. The method according to claim 1, wherein the chip further comprises at least one electrical connection pad on its back side.

11. An ultrasound probe comprising a flexible interconnection substrate and a plurality of elementary chips bonded to a surface of the flexible interconnection substrate, each elementary chip comprising one or a plurality of ultrasound transducers, each elementary chip comprising a front side comprising at least one electrical connection pad and a back side,
wherein a portion of the flexible interconnection substrate is folded over the elementary chips, so that said surface of the flexible interconnection substrate extends over and is bonded to at least a portion of the back side of each elementary chip and at least a portion of the front side of each elementary chip, said at least one electrical connection pad of the front side of each elementary chip being connected to a corresponding electrical connection pad of said surface of the interconnection substrate.
